(19) United States
(12) Patent Application Publication
(10) Pub. No.: US 2006/0234966 A1
(43) Pub. Date: Oct. 19, 2006

(54) Method for expressing inducible rna [in cells, nucleic acid molecules therefor and cells transformed by said molecules

(76) Inventors: Annick Harel-Bellan, Paris (FR); Lauriane Fritsch, Maison (FR); Redha Sekhri, Cachan (FR)

Correspondence Address: IP GROUP OF DLA PIPER RUDNICK GRAY CARY US LLP, 1650 MARKET ST, SUITE 4900, PHILADELPHIA, PA, 19103, US

(21) Appl. No.: 10/535,692

(22) PCT Filed: Nov. 21, 2003
(86) PCT No.: PCT/FR03/03447

§ 371 Date: Sep. 14, 2005

(30) Foreign Application Priority Data
Nov. 21, 2002 (FR) 02/14600

(51) Int. Cl. A61K 48/00; C07H 21/04

(52) U.S. Cl. 514/44; 435/455; 536/23.2; 435/325

(57) ABSTRACT

A method of expressing RNAi in cells including introducing into cells a molecule of nucleic acid including the sequence sense and antisense of RNAi placed under the control of a promoter of single transcription, which sense and antisense sequences are separated by a sequence of DNA including a sequence for the stop of this transcription, which DNA sequence is framed at each of its ends by a lox site, and (ii) placing Cre in contact of the lox sites to obtain by site-specific recombination the elimination of the DNA sequence and the stop sequence of the transcription such that the sense and antisense sequences are no longer separated except by the remaining lox sequence and, thus, permit transcription of the RNAi in its entirety with the residual lox sequence as a loop.

The invention relates to the area of biology and more especially to the preparation of double-stranded oligonucleotides for use in a process of RNA interference (RNAi or ARNi).

RNA interference, also designated “siRNA” or “RNAi” or also co-suppression, has been demonstrated in plants, where it was observed that the introduction of a long double-stranded RNA corresponding to a gene induces the specific and efficacious repression of the target gene. The mechanism of this interference comprises the degradation of the double-stranded RNA into short duplexes of oligonucleotides of 20 to 22 nucleotides.

RNA interference has now been applied to mammals for specifically inhibiting genes for functional genetic applications. In fact, siRNAs permit the identification of the function of genes demonstrated by the sequencing of the human genome, either in models of cellular culture or in animal models, in particular in the mouse. RNA interference is also useful in the therapeutic area for the treatment or prevention of cancers, infectious diseases and, more generally, diseases involving a heterologous or homologous mutated gene (S. M. Elbashir, J. Harborth, W. Lendeckel, A. Yalcin, K. Weber and T. Tuschl (2001a). Duplexes Of 21-Nucleotide RNAs Mediate RNA Interference in Cultured Mammalian Cells. Nature 411, pp. 494-498; S. M. Elbashir, J. Martinez, A. Patkaniowska, W. Lendeckel and T. Tuschl (2001b). Functional Anatomy of siRNAs for Mediating Efficient RNAi in Drosophila Melanogaster Embryo Lysate. Embo J 20, 6877-6888).

The siRNAs are short sequences of double-stranded RNA that can be introduced in the form of synthetic oligonucleotides or in the form of plasmid permitting their transcription.

The use of plasmids has numerous advantages, in particular for functional genetic applications. It permits the expression of double-stranded RNA in a stable manner in the cells and thus the easier inhibition of proteins with a long half-life. In fact, synthetic siRNAs have a half-life of 3 days in mammalian cells. It also permits long-term effects to be analyzed. On the other hand, it requires the establishing of lines expressing the construction in a stable manner, which has several disadvantages. In particular, it is necessary to compare stable lines with each other, which is generally difficult to interpret because the cellular lines drift. On the other hand, it is impossible to study the proteins indispensable for the cell, since their inhibition would block the proliferation of the cells and thus prevent the establishing of the stable line. It is therefore indispensable to be able to induce at will the expression of the siRNA.

The invention has the specific problem of palliating these disadvantages by making available a system for expressing an siRNA in a stable and inducible manner. This problem is solved by the use of the CRE-lox system for the expression of an siRNA in mammalian cells. The invention thus relates to a method of expressing RNAi in cells comprising:

The introduction into eukaryotic cells of a molecule of nucleic acid comprising the sequences sense and antisense of RNAi placed under the control of a promoter of single transcription, which sense and antisense sequences are separated by a sequence of DNA comprising a sequence for the stop of this transcription, which DNA sequence is framed at each of its ends by an lox site,

The placing in contact of the lox sites with Cre in order to obtain by site-specific recombination the elimination of the DNA sequence and of the stop sequence of the transcription in such a manner that that these sense and antisense sequences are no longer separated except by the remaining lox sequence and thus permit the transcription of the RNAi in its entirety with the residual lox sequence as loop.

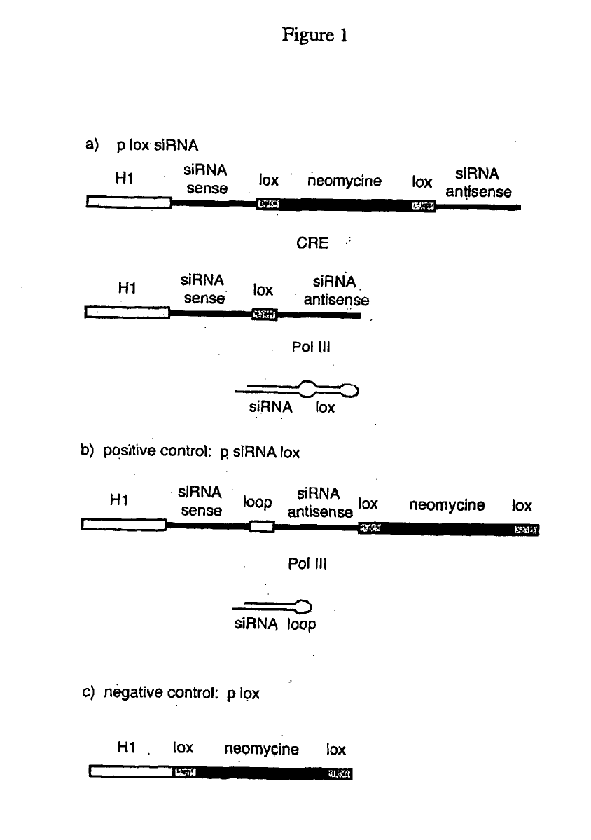

According to a particular realization of the method of the invention said molecule of nucleic acid comprises 5′ toward 3′, as shown in FIG. 1, a transcription promoter compatible with said cells, the sense sequence of the RNAi, a first lox site, a DNA sequence comprising a transcription terminator, the second lox site and the antisense sequence of the RNAi.

This molecule of nucleic acid is advantageously a plasmid. It can also be a retrovirus.

The cells transfected with this molecule of nucleic acid are mammalian cells. The method can be applied as well to the transfection of cells in culture as directly to the animal.

In fact, the invention permits the reliable analysis of human genes from a functional viewpoint in cells in culture or in animals and in particular in mice. In fact, there are systems permitting the inducible expression of CRE in cells and in animals. In the mouse, the CRE can also be expressed in a tissue-specific manner permitting the inactivation of a gene specifically in these tissues.

The CRE can be placed in contacts with the lox sites via the transfection of the cells for the molecule of nucleic acid comprising a regulatory sequence and the cre gene.

The DNA sequence separating the sense and antisense sequences of the RNAi and comprising the transcription terminator is advantageously a gene resistant to an antibiotic such as neomycin thus permitting in addition the selection of the transfected cells.

The invention also relates to a molecule of nucleic acid described above for implementing the method of the inducible expression of RNAi in cells.

The invention also relates to a cell or a cell line transfected by a molecule of nucleic acid previously described and to the animals whose cells were transfected by this molecule of nucleic acid. Finally, the invention relates to compositions, especially pharmaceutical compositions comprising as active substance at least one molecule of nucleic acid above or cells transformed by it possibly associated in the composition with a compatible excipient.

Other advantages and characteristics of the invention will become apparent from the following examples that make reference to the attached drawings.

FIG. 1 represents the strategy for the expression of siRNAs in an inducible manner in accordance with the invention.

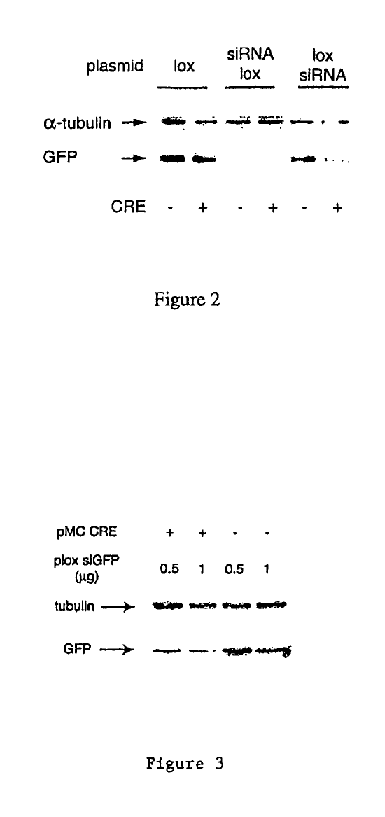

FIGS. 2, 3 represent the induction of the activity of the RNAi by the CRE.

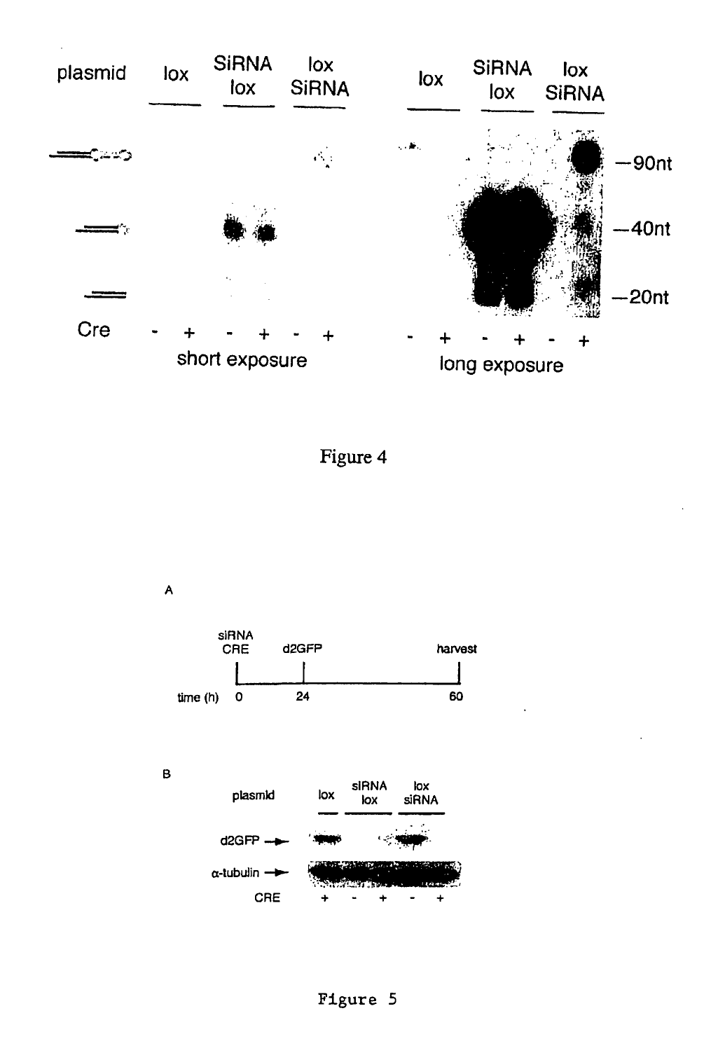

FIG. 4 represents the inhibition of the GFP marker by the RNAi.

FIG. 5 represents the inhibition of the GFP marker as a function of CRE during the course of transfection in two stages.

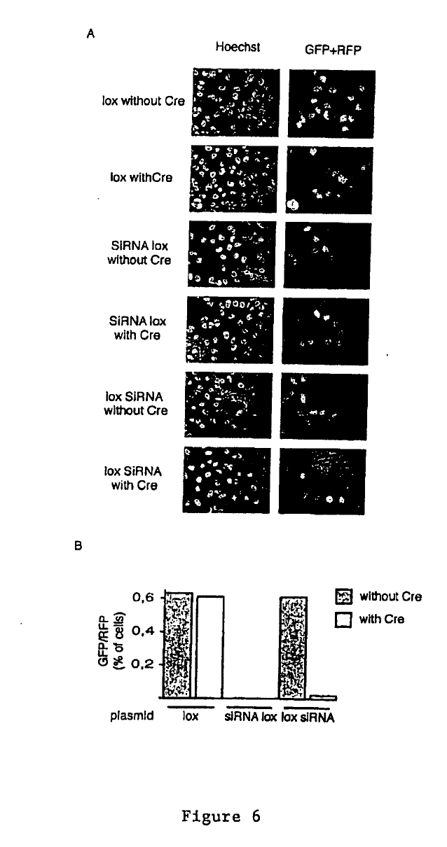

FIG. 6 represents the inhibition by RNAi of the GFP marker integrated in a cell line.

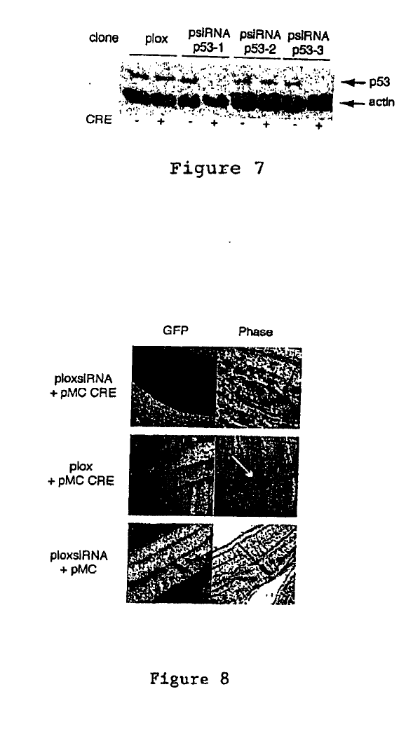

FIG. 7 represents the inhibition by RNAi of the endogenous gene p53 with establishment of stable cell lines.

FIG. 8 represents the activity in vitro of RNAi.

### EXAMPLE 1

The plasmid plox siRNA comprises a promoter Pol II controlling a gene resistant to an antibiotic, neomycin. The neomycin cassette is surrounded by lox sites. In a first phase a promoter Pol III (H1) was inserted in the direction opposite to promoter Pol II. The promoter H1 introduced into the plasmid behind the second region loxp with the restriction enzymes Nhe1 and XbaI is obtained by PCR from the following primers:

(SEQ ID NO. 1)

5′ CTAGCTAGCCCATGGAATTCGAACGCTGACGTC 3′
Forward

(SEQ ID NO. 2)

5′ GCTCTAGAGTGGTCTCATACAGAACTTATAAGATTCCC 3′
Reverse

This plasmid is based on the plasmid pSUPER permitting the constitutive expression of siRNA and described by Brummelkamp et al.

The DNA sequences corresponding to the siRNA sense were then introduced immediately after the H1 promoter at the level of the XbaI site.

siRNA sense:

(SEQ ID NO. 3)

5′ CTAGACCCGCAAGCTGACCCTGAAGTTCATT 3′

siRNA complementary sense:

| (SEQ ID NO. 4) | |
|---|---|
| | 5′ CTAGAATGAACTTCAGGGTCAGCTTGCGGT 3′ |

Finally, the DNA sequences corresponding to the antisense siRNA were introduced following the second loxp region at the level of the Bamh1 and Kpn1 sites.

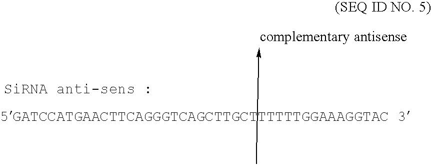

SiRNA antigens complémentaire:

(SEQ ID NO. 6)

5′ CTTTCCAAAAAAGCAAGCTGACCCTGAAGTTCATG 3′.

The psiRNA lox is obtained by inserting the entire DNA sequence of the siRNA directly after the promoter HI at the level of the XbaI sites. The sense and antisense siRNAs are separated by a loop.

SiRNA:

(SEQ ID NO. 7)

5′ CTAGTTTCCAAAAAAGCAAGCTGACCCTGAAGTTCATTCTCTTGAAA
TGAACTTCAGGGTCAGCTTGCGGGT 3′

SiRNA complémentaire:

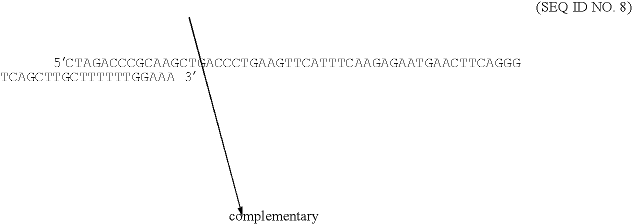

Mammalian cells COS-7 were transfected with the polyfect (Qiagen) with 4 μg of expression vectors of the siRNA (plox siRNA, psiRNalox or plox) as indicated and a vector expressing the CRE recombinase or the corresponding empty vector (8 μg) as well as a vector for the expression of green fluorescent protein or GFP (500 ng). Sixty hours after the transfection a Western blot was performed starting from the total extracts in using an antibody directed against the GFP (Santa cruz) or the cellular tubulin (Sigma) in order to evaluate the quantity of proteins used for this test (FIG. 2). Fibroblast cells (3T3) were transfected with 0.5 μg or 1 μg of the expression vector plox siRNA as indicated in FIG. 3 and a vector expressing the CRE recombinase or the corresponding empty vector (8 μg) as well as a vector for the expression of GFP (500 ng). Sixty hours after the transfection a Western blot was performed starting from the total extracts using an antibody directed against the GFP (Santa cruz) or the cellular tubulin (Sigma) in order to evaluate the quantity of proteins used for this test (FIG. 3).

In the absence of CRE the two constituent parts of the siRNA (sense and antisense strand are separated by the neomycin gene, that comprises a transcription stop sequence for Pol III. Under these conditions only the sense strand of the siRNA is transcribed and the siRNA is inactive: The target protein is normally expressed as shown in FIG. 2 and in FIG. 3, third and fourth line. In the presence of CRE the plasmid undergoes a process of recombination in the cell yielding a product in which the neomycin sequence is eliminated and in which the two 12 siRNAs are only separated by the remaining lox sequence, in which there is no transcription stop sequence for Pol III. The siRNA is therefore transcribed in its entirety with the residual lox sequence that serves as “loop”. This siRNA is active and the target protein is inhibited (compare line 1 or 2 with line 3 or 4, FIGS. 2,3).

The inhibition is closely linked to the activity of the siRNA since in the presence of CRE the inhibition is only observed in the presence of the complete siRNA and not in the presence of the vector for the expression of the empty siRNA (line 1). Its activity is equivalent to that of an siRNA serving as positive control, expressed in a constitutive manner (because the entirety of the sequence from which it is transcribed is placed in front of the neomycin gene).

On the other hand, the analysis by Northern shows the processing of the precursor and the synthesis of the siRNA induced by the CRE (FIG. 4). The total RNA of the COS-7 cells was extracted after 60 h of transfection then analyzed by northern blot with a probe marked at 32P directed against the antisense strand of the siRNAs produced:

(SEQ ID NO. 9)

5′ CTTTCCAAAAAAGCAAGCTGACCCTGAAGTTCATG 3′

FIG. 4 shows that the inhibition is closely linked to the expression of the siRNA induced by the CRE.

### EXAMPLE 2

1) Methods

Plasmid constructions were realized in accordance with the method presented in example 1. The plox vector was constructed by inserting the promoter Pol III (HI) into the plasmid ploxNeo as an Nhe1-Xba insert. The sequences corresponding to the sense and antisense siRNA strands were introduced as synthetic oligonucleotides respectively using the restriction sites XbaI or BamH1 and Kpn.

SiRNA sens:

(SEQ ID NO. 10)

5′ CTAGCCCCGCAAGCTGACCCTGAAGTTCATT 3′

SiRNA anti-sens:

(SEQ ID NO. 11)

5′ GATCCATGAACTTCAGGGTCAGCTTGCTTTTGGTACCTAGACCC 3′

This vector will be used in the subsequent examples.

Mammalian cells COS-7 were transfected in two stages. The first transfection was realized with 1 μg vectors for the expression of the siRNAs (plox siRNA, psiRNAlox or plox) and 2 μg of a vector expressing CRE recombinase or the corresponding empty vector. Twenty-four hours after this first transfection the cells were transfected with 500 ng of a vector for the expression of CMV-d2GFP (Clontech). A Western blot was realized in conformity with example 1.

1) Results

FIG. 5 shows that GFP is undetectable during a transfection in two stages in the course of which the siRNA was able to form 24 hours before the transfection of the vector for the expression of GFP.

### EXAMPLE 3

1) Methods

HeLa cells 1002 (cell line derived from the HeLa cells possessing an integrated transgene coding for the GFP inducible by doxocycline) were transfected with 300 ng vectors for expressing CMV luc or CMV-RFP, with 8 μg of vector expressing CRE recombinase or the corresponding empty vector and with 4 μg of vectors for expressing siRNAs (plox siRNA, psiRNAlox or plox). Seventy-two hours after the transfection the cells were treated for twenty-four to seventy-two hours with doxycycline (1 μg/ml) before observation under an Axiovert fluorescent microscope.

2 Results

FIG. 6 shows the activity of siRNA on the expression of a GFP marker gene integrated into the genome of the cell line and inducible by doxycycline. The expression of the marker is observed in approximately 30% of the cells twenty-four hours after induction (FIG. 6A) and in 60% of the cells forty-eight hours after induction (FIG. 6B). Similar proportions are observed in the cells for the control of the transfection (positive RFP) transfected with the empty plox vector (FIGS. 6A, 6B).

Independently of the expression of the CRE protein, no expression of the GFP target protein is observed in the cells transfected with the psiRNA plox vector expressing the siRNA in a constitutive manner. In the absence of CRE, among the cells transfected with the vector plox siRNA the proportion of positive GFP cells is approximately 30% one day after induction and approximately 65% two days after induction, but this proportion is less than 5% in the presence of CRE.

### EXAMPLE 4

1) Methods

A plox siRNA expression vector directed against p53 (plox siRNA tp53) was constructed in accordance with the method presented in example 2. The sense and antisense siRNA sequences used were the following:

SiRNA sens:

5′ GCATGAACCGGAGGCCCATT 3′ (SEQ ID NO. 12)

SiRNA anti-sens:

5′ GATCCATGGGCCTCCGGTTCATGC 3′ (SEQ ID NO. 13)

U20S cells were transfected either with the empty vector plox or with a plox siRNA expression vector directed against gene p53 (plox siRNA p53). Stable clones were established by virtue of the neomycin selection marker. These stable clones were then transfected either with a vector expressing CRE recombinase or with the corresponding empty vector pMC, then selected with the aid of a different selection marker (hygromycin). The expression of the pMC was checked four weeks later by Western blot.

1) Results

FIG. 7 shows three examples of clones transfected with the plox siRNA vector p53 presenting an inhibition of p53 dependent on CRE. No inhibition was observed in the clones ploxsiRNAp53 that were transferred with the empty vector pMC, as well as in the clones transfected in a stable manner with the empty vector plox. FIG. 7 shows the cell lines transfected in a stable manner in which a target endogenous gene is inhibited.

### EXAMPLE 5

1) Methods

The construction MCK-nlslacZ contains the sequence coding for nuclear β-galactosidase under the control of the promoter of muscle kinase creatine. The use of such an expression vector permits the marking of the nuclei of the transfected muscular fibers. The other expression vectors used are: The vector for the expression of CRE or the corresponding empty vector, the vector for expressing plox, and the vector for expressing plox siRNA.

Transgenic actin GFP mice of five to six weeks (Ikawa et al.) were anaesthetized with 300 μl of 0.05% xyalazine-1.7% ketamine in NaCl 0.9%. After incision of the skin, 8 μg of DNA containing 3 μg of vectors for the expression of CRE and/or of the siRNAs and 2 μg of MCK-mlslacZ were injected into the tibialis anterior muscle (TA) with the aid of a 1 ml syringe provided with a 27 caliber needle. Caliper electrode plates (Q-biogen, France) are immediately applied on each side of the muscle and a series of eight electric pulses (2 Hz, 20 ms each) is delivered with a standard electroporator with a squarewave signal (ECM 830, Q-biogen). The electrical contact is ensured by the application of a conductive gel. Twelve days after the injection the TA muscles are dissected and fixed on paraformaldehyde at 4% in PBS buffer (saline phosphate buffer) then incubated for two to three hours in 5-bromo-4-chloro-indol-β-galactoside 0.4 mg/ml, K3Fe (CN) 6 4 mM, K4Fe(CN) 6 4 mM and MgCl2 2 mM, PBS at 37° C. for the lacZ coloration. The positive LacZ regions are then dissected under the microscope. The images of fluorescence and in phase contrast are obtained with a Zeiss confocal microscope (LSM510, Zeiss).

Results

FIG. 8 shows that the combination of the plasmid expressing CRE and of the plox siRNA GFP vector induces a marked reduction of the expression 1a GFP in the transfected fibers (the arrow indicates the positive LacZ nuclei). The expression of CRE in the presence of the plox control vector as well as the transfection of the ploxsiRNA vector in the absence of CRE do not affect the expression of GRP in the transfected fibers. FIG. 8 shows that the expression of the siRNA induced by CRE can reduce the expression of a gene in vivo.

1-9. (canceled)

10. A method of expressing RNAi in cells, comprising:
introducing into eukaryotic cells a molecule of nucleic acid comprising sense and antisense sequences of RNAi placed under control of a promoter of single transcription, the sense and antisense sequences being separated by a sequence of DNA comprising a sequence for stopping transcription, wherein the DNA sequence is framed at each end thereof by a lox sit, and
placing Cre in contact with the lox sites to obtain by site-specific recombination elimination of the DNA sequence and the stop sequence of the transcription such that the sense and antisense sequences are no longer separated except by a remaining lox sequence and thereby permit transcription of the RNAi in its entirety with the remaining lox sequence as a loop.

11. The method according to claim 10, wherein the molecule of nucleic acid comprises from 5′ into 3′, a transcription promoter compatible with the cells, the sense sequence of the RNAi, a first lox site, a DNA sequence comprising a transcription terminator, a second lox site and an antisense sequence of the RNAi.

12. The method according to claim 10, wherein the molecule of nucleic acid is a plasmid.

13. The method according to claim 10, wherein the transfected cells are mammalian cells.

14. The method according to claim 10, wherein the DNA sequence separating the sense and antisense sequences of the RNAi and comprising the transcription terminator is a gene resistant to an antibiotic.

15. The method according to claim 14, wherein the antibiotic is neomycin.

16. The method according to claim 10, wherein the cells are also transfected with a molecule of nucleic acid comprising a regulating sequence and the cre gene.

17. A molecule of nucleic acid comprising sense and antisense sequences of RNAi placed under control of a promoter of single transcription, the sense and antisense sequences being separated by a sequence of DNA comprising a sequence for stopping transcription, wherein the DNA sequence is framed at each end thereof by a lox site.

18. A cell or a cell line transfected by the molecule of nucleic acid in accordance with claim 17.

19. A pharmaceutical composition comprising a therapeutically effective amount of an active substance of at least one molecule of nucleic acid in accordance with claim 17 and a compatible excipient.

20. A pharmaceutical composition comprising a therapeutically effective amount of an active substance of at least a cell or cell line in accordance with claim 8 and a compatible excipient.


SEQUENCE LISTING (13 sequences)

SEQ ID NO: 1 - DNA, 33 bp, Artificial Sequence
  Description of Artificial Sequence Synthetic primer
ctagctagcccatggaattcgaacgctgacgtc

SEQ ID NO: 2 - DNA, 38 bp, Artificial Sequence
  Description of Artificial Sequence Synthetic primer
gctctagagtggtctcatacagaacttataagattccc

SEQ ID NO: 3 - DNA, 31 bp, Artificial Sequence
  Description of Artificial Sequence Synthetic DNA oligonucleotide transcribing siRNA
ctagacccgcaagctgaccctgaagttcatt

SEQ ID NO: 4 - DNA, 30 bp, Artificial Sequence
  Description of Artificial Sequence Synthetic DNA oligonucleotide transcribing siRNA
ctagaatgaacttcagggtcagcttgcggt

SEQ ID NO: 5 - DNA, 43 bp, Artificial Sequence
  Description of Artificial Sequence Synthetic DNA oligonucleotide transcribing siRNA
gatccatgaacttcagggtcagcttgcttttttggaaaggtac

SEQ ID NO: 6 - DNA, 35 bp, Artificial Sequence
  Description of Artificial Sequence Synthetic DNA oligonucleotide transcribing siRNA
ctttccaaaaaagcaagctgaccctgaagttcatg

SEQ ID NO: 7 - DNA, 72 bp, Artificial Sequence
  Description of Artificial Sequence Synthetic DNA oligonucleotide transcribing siRNA
ctagtttccaaaaaagcaagctgaccctgaagttcattctcttgaaatgaacttcagggtcagcttgcgggt

SEQ ID NO: 8 - DNA, 72 bp, Artificial Sequence
  Description of Artificial Sequence Synthetic DNA oligonucleotide transcribing siRNA
ctagacccgcaagctgaccctgaagttcatttcaagagaatgaacttcagggtcagcttgcttttttggaaa

SEQ ID NO: 9 - DNA, 35 bp, Artificial Sequence; identical to SEQ ID NO: 6
  Description of Artificial Sequence Synthetic DNA oligonucleotide transcribing siRNA

SEQ ID NO: 10 - DNA, 31 bp, Artificial Sequence
  Description of Artificial Sequence Synthetic DNA oligonucleotide transcribing siRNA
ctagccccgcaagctgaccctgaagttcatt

SEQ ID NO: 11 - DNA, 44 bp, Artificial Sequence
  Description of Artificial Sequence Synthetic DNA oligonucleotide transcribing siRNA
gatccatgaacttcagggtcagcttgcttttggtacctagaccc

SEQ ID NO: 12 - DNA, 20 bp, Artificial Sequence
  Description of Artificial Sequence Synthetic DNA oligonucleotide transcribing siRNA
gcatgaaccggaggcccatt

SEQ ID NO: 13 - DNA, 24 bp, Artificial Sequence
  Description of Artificial Sequence Synthetic DNA oligonucleotide transcribing siRNA
gatccatgggcctccggttcatgc